(19) United States
(12) Patent Application Publication
(10) Pub. No.: US 2006/0058586 A1
(43) Pub. Date: Mar. 16, 2006

(54) Method and apparatus for transitioning a diet

(76) Inventor: David R. Humble, Deerfield Beach, FL (US)

Correspondence Address: EDWARDS & ANGELL, LLP, P.O. BOX 55874, BOSTON, MA, 02205, US

(21) Appl. No.: 10/939,653

(22) Filed: Sep. 13, 2004

(51) Int. Cl. A61B 5/00

(52) U.S. Cl. 600/300; 128/920

(57) ABSTRACT

A system stores nutritional information for a variety of foods. A user, interactively connected to the system, via the Internet or the like, accesses the system. The system stores user characteristic data including current weight, diet characteristic information. The dieter inputs their daily food intake to the system. The system monitors the natural diet over a predetermined time, the length of time being sufficient to determine the natural eating patterns of the individual. As a function of the goal, the server ranks the foods eaten in order of worst to best for achieving the goal. The system compares the worst food to foods of the same type and suggests an alternative food to be included in the diet, which is more in keeping with the diet goal. This process is then repeated until the diet is sufficiently modified to achieve the diet goal.

### BACKGROUND OF THE INVENTION

1. Field of the Invention

This invention is directed to a method for dieting, and more particularly, a method and apparatus for transitioning from one diet to another.

2. Background

By the time people reach adulthood, they have established their own natural personal diet which is a function of what they eat, how much of each thing they eat, and even when they eat it. It is also very common for an adult to be placed on a diet. An adult may be placed on a diet for a variety of reasons, the most common of which is weight loss. However, people also diet for weight gain and muscle mass, for health reasons such as lowering cholesterol or sodium intake, or for cultural reasons such as a vegetarian diet.

To facilitate dieting, it is now known in the art that web portals, such as www.ediets.com, provide interactive software engines which receive personal information about the potential dieter such as the dieting goals, and then create a menu of meals to help the dieter attain the goals. Furthermore, the menu may be governed by the type of diet the dieter desires, such as low-carb as opposed to low-fat, as opposed to high-protein, as opposed to low-calorie, or Kosher, or allergic to dairy or the like. The menu may also be governed by medical considerations such as Type 2 diabetes, high blood pressure, lactose intolerance and the like.

Although a significant portion of the population is dieting on any given day, many people have a hard time keeping with a diet resulting in what is known as the “yo-yo” effect. People strictly adhere to a diet and obtain the results, such as weight loss, then drop the diet and return close to their original position, and then perform corrective dieting again. In some cases, there is no “yo-yo” and the person never is able to maintain the diet and merely gives up.

Accordingly, what is desired is a method of dieting, which avoids the inevitable dropping of a new diet and a system for making it possible.

### SUMMARY OF THE INVENTION

A system stores nutritional data for a variety of foods. A user, interactively connected to the system, via the Internet or the like, accesses the system. This system stores user characteristic data including current weight and diet characteristic data. The dieter inputs their daily food intake into the system. The system monitors the natural diet over a predetermined time, the length of time being sufficient to determine the natural eating patterns of the individual. As a function of the goal, the system ranks the foods eaten in order of worst to best for achieving the dieting goal. The system compares the worst food to the nutritional data for foods of the same type and suggests an alternative food to be included in the diet, which is more in keeping with the diet goal. This process is then repeated until the diet is sufficiently modified to achieve the diet goal.

In a preferred embodiment, the system establishes the food to be substituted by applying a series of rules as a function of the diet goal. The diet goal may be one or more of fewer calories, lower carbohydrates, lower cholesterol, lower sodium, lower fat, or the like. The system ranks the eaten foods in relation to the person's objectives and described conditions. It then periodically suggests one or two alternative foods for the dieter to choose from to replace the worst ranked foods and, over time, the natural diet is modified in a way that the mental and physical challenges are avoided.

### BRIEF DESCRIPTION OF THE DRAWINGS

For a fuller understanding of the invention, reference is had to the following description taken in connection with the accompanying drawings in which:

FIG. 1 is a schematic diagram of a system for providing the on-line transitioning of a diet in accordance with the invention; and

FIG. 2 is a flow diagram of the method for transitioning a diet in accordance with the invention.

### DETAILED DESCRIPTION OF THE INVENTION

This invention provides for a system and method for transitioning users onto a diet in a manner that increases the likelihood that they remain with the diet. Applicant has noted that one of the reasons that diets are difficult to maintain is the system shock of immediately transitioning from one's natural diet to a brand-new diet. This is because a new diet is a significant mental and, in some cases, physical challenge that most people cannot manage. It causes a physical reaction, such as cravings for forbidden foods, and a mental reaction as a result of the dieter being forced to forego many of the foods which have formed a staple of their diet, and replace them with other foods; to some of which they are not accustomed. People become “addicted” to their natural diet. When forced to go “cold turkey” with a new diet many natural diet items may be unnecessarily removed from the diet increasing the “shock” effect of the new diet to the dieter.

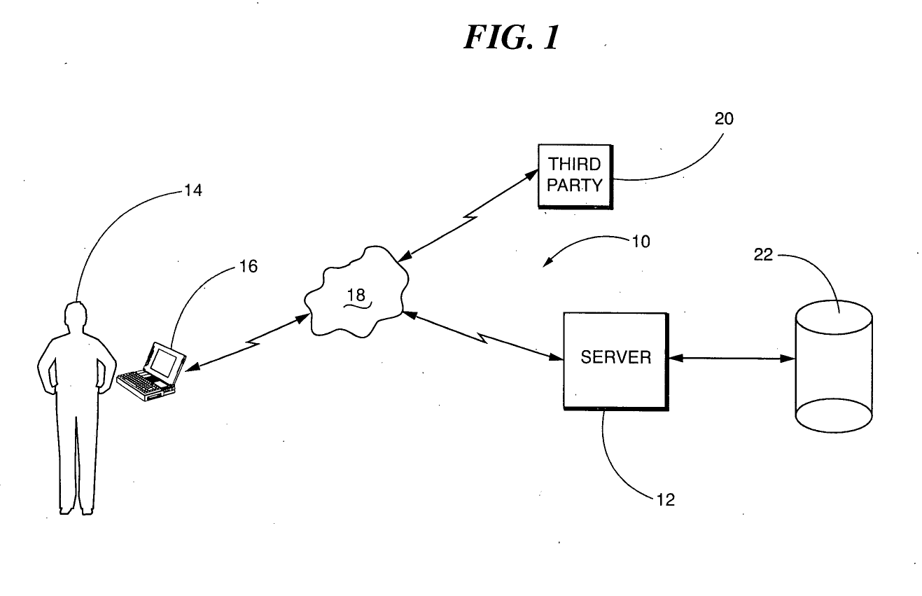

Reference is made to FIG. 1 in which a system, generally indicated as 10, is provided. System 10 includes a server 12 operatively communicating with a database 22. Server 12 communicates with users (“dieters”) 14 at a dieter computer 16 through Internet 18. Server 12 may also communicate with third-party databases 20 through Internet 18. In a preferred embodiment server 12 provides an interactive web-based portal such as a web page for interacting with dieter 14. It should be noted that computer 16 may be any interactive device which allows dieter 14 to communicate with server 12. It should be noted, the preferred embodiment is an Internet based system. However, the system may include any computing device capable of calculating a worst food and a replacement food as will be described below. The computing device can communicate with a dieter 14 by Internet, radio frequency, telephone pager, or personal direct input by way of non-limiting example.

Database 22 stores diet goal characteristic data, a set of diet rules, menu data, and nutritional data for foods stored in database 22. It should be noted, that in alternative embodiments, any and all of this data can be stored at an accessible third-party database 20 which may be accessed by server 12 over Internet 18. By way of example, nutritional information may be stored in database 22 or may be accessed at a third party database 20 such as the USDA website by way of non-limiting example.

Dieter characteristic data may include any combination of a dieter's weight, height, age, dieting goal as discussed above, dieting preferences as discussed above, and activity level. The stored diet rules are the logic rules for determining which foods bring dieter 14 closer to or maintain dieter 14 within their dieting goal. Menu data are food arrangements grouped as meals as determined by server 12. Nutritional data is the nutritional information, such as the information found on food packaging for the respective foods which make up the foods grouped in the menu data. It should be noted, that menu data can be a group of combined foods such as a hamburger with lettuce, tomato and condiments or a single item of food in the case of snacks such as peanuts or carrot sticks. The nutritional data may be presented as the nutritional data for each constituent of the hamburger or, the hamburger as a whole as a function of portion size. Any or all of this data may be accessed by server 12 either at database 22 or third-party data source 20.

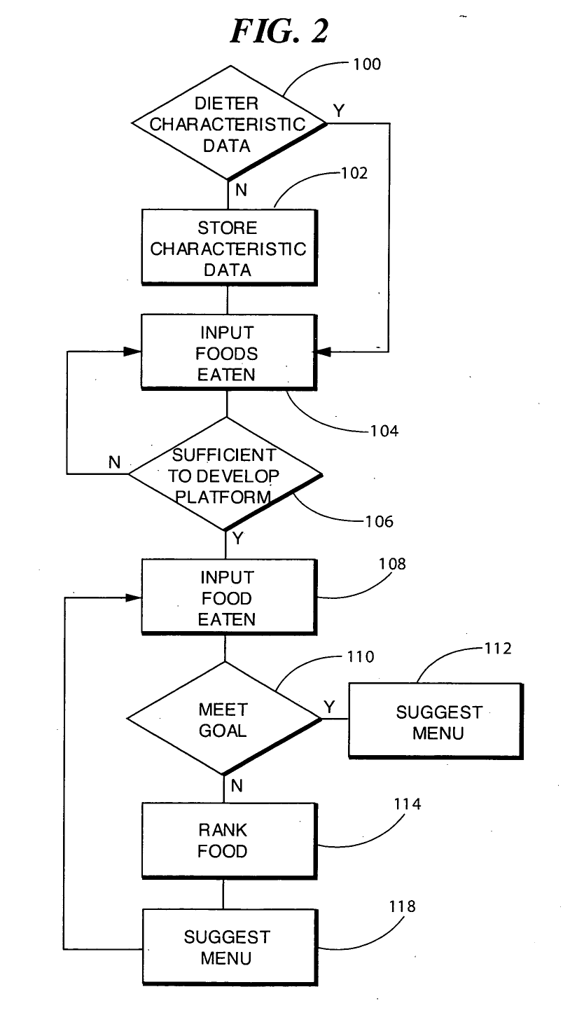

Reference is now made to FIG. 2 in which a flow diagram for transitioning a diet in accordance with the invention is provided. In a first step 100 it is determined whether or not dieter characteristic data for a dieter 14 is known to server 12 as stored in database 22. If no, then dieter characteristic data is stored in step 102. In the preferred embodiment, dieter 14 inputs dieter characteristic data at computer 16 which is transmitted to server 12 over Internet 18 for storage in database 22. However, it should be noted that other communication means, such as telephone, direct input, or other non-Internet communication may be used to enter the information.

Furthermore, server 12 may be any device capable of storing data, processing data and communicating with remote parties. Dieter characteristic data would consist of a dieter ID, either name, e-mail address, or identification number such as social security number. It would also include the dieter goal such as weight loss, weight gain, weight maintenance with lowered cholesterol, sodium, fat or carbohydrate intake, or any other goal which can be controlled by the intake of specific food types having characteristics which can be tracked. The height, weight and activity level of dieter 14 may also be stored as dieter characteristic data. Medical conditions such as high blood pressure, high cholesterol, Type I diabetes or the like, and other medical information that dieter 14 consider important will be stored as characteristic data and be used to create menus as discussed below.

In a step 104 dieter 14 keeps track of the types and quantities of food they have eaten by inputting food eaten to server 12. By way of example, dieter 14 has a fast-food lunch of a hamburger such as a Whopper®, a Quarter-Pounder®, or an unbranded hamburger, with french fries and a diet coke. Dieter 14 enters that information into server 12. Server 12 checks database 22 which, as discussed above, includes dietary information such as the ESHA Research Library (ESHA data) or the USDA's Nutrition Library. These databases include nutritional information for commonly known foods, and the components for such commonly known foods. Therefore, the nutritional information such as minerals, vitamins, grams of fat, fiber, carbohydrates, sugars and calories for a Quarter-Pounder® are known. Assuming, that they were not known, then the user would input quarter-pound hamburger with bun, tablespoon of mayonnaise, ketchup, mustard (as appropriate), leaf of lettuce, slice of tomato, etc., 8 ozs. of french-fried potatoes, and 12 ozs. of diet cola. This information would be stored at database 22 along with its nutritional information as determined from database 22.

In a step 106 it is determined whether or not there are sufficient entries from step 104 to determine dieter 14's natural diet. If dieter characteristic data is already stored as determined in step 100, then dieter 14 proceeds directly to step 104 and inputs eaten food to server 12. Server 12 determines, based upon a sampling, whether or not there is statistically significant information to determine the natural diet of dieter 14. If not, steps 104 and 106 are repeated on a periodic basis until there is sufficient information to determine the natural diet of dieter 14. If there is insufficient information to develop the pattern of the natural diet, then steps 104 and 106 are repeated until there is a significant sample size to determine dieter 14's natural diet. For the purposes of this invention, natural diet corresponds to the average daily nutritional intake of dieter 14 prior to any proposed change in the diet.

If the natural diet has been determined in a step 106, in a step 108, dieter 14 inputs food eaten, or if, as in the first time the natural diet is determined, and the food eaten has been input in a step 104 that data is used by server 12. In a step 110, server 12 determines whether the input food eaten is within the parameters of the dieting goal of dieter 14 as determined from dieter characteristic data. By way of non-limiting example, if weight loss is desired by way of a low-calorie diet, and the input food corresponds to the fast-food hamburger meal discussed above, then server 12 will determine the calories and other nutritional information from database 22, and compare these values to a model nutritional value corresponding to a menu for accomplishing the diet goal. If in fact, the calorie limit or other nutritional parameters have not been exceeded, then in a step 112, as known in the art, a menu as determined from the dieter characteristic data which maintains the dieting goal of dieter 14 will be suggested to dieter 14 by server 12. Server 12 will provide menus, meal by meal, day by day, week by week, and will present them either meal by meal or across any predetermined time period to dieter 14 at computer 16.

Server 12 determines the extent to which each eaten food is in keeping with the diet goal. In a preferred embodiment, if server 12, based upon the nutritional data corresponding to the input food, determines in step 110 that the eaten food does not accomplish the dieting goal as determined from dieter characteristic data, then server 12 will rank the components of each meal.

The rank could be from most in keeping with the desired goal (best) to least in keeping with the desired goal (worst) or vice versa. The ranking is determined by rules stored in database 22 for operation by server 12. Based upon the input food, and the diet goals as determined from step 104 and the dieter characteristic data, server 12, utilizing the nutritional information for food stored in database 22 will determine which eaten items are outside the healthy parameters for the diet goal, such as high in calories, high in fat, high in carbohydrate, sodium, cholesterol, or the like.

Staying with the example of the low-calorie, low-fat diet, server 12 will identify those food items eaten which are outside the healthy parameters. Server 12 will determine which foods are lowest in calories and fat, therefore driving dieter 14 towards their goal diet, and which of those are highest in calories and fat; driving dieter 14 away from their goal and rank the foods accordingly. In step 114, the food is ranked as discussed above.

The food is ranked in accordance with the rules stored in database 22. In the preferred embodiment there will be hundreds of rules, however, for ease of description, and by way of example only, they can be represented in this example by five basic example categories namely; (1) rules on calories; (2) rules on carbohydrates; (3) rules on fats; (4) rules on cholesterol; (5) rules on sodium. It should be noted that there may even be more specific rules such as rules with respect to sodas, red meats, vegetarian or the like corresponding to aspects of the diet as stored as a dieter 14's characteristic data. By way of example, the rules may provide more granularity by determining whether or not the fat intake is good fats as opposed to bad fats, or whether the diet is nutritionally sound and requires an increase in vegetables, fruit or minerals above and beyond the overall goal of lower fat and lower calories.

Server 12 then utilizes logic as a function of the diet goal such as in our example weight loss through low fat, and low calories and gives each of the rules different weight. For example, a low-carbohydrate diet may give more weight to the low-calorie and low-carbohydrate rules in creating a diet than it gives to a rule concerning the intake of red meat as a low-carbohydrate diet is substantially protein insensitive. On the other hand, as in our example, loss of weight through low-calorie and low-fat intake, the rules on fat may be given highest priority, followed by the rules on calories, and the rules on carbohydrates may be given significantly lesser weight when applied to determining a suggested menu.

Once the foods have been ranked, in step 116, the rules are applied to the menu of ranked foods. For example, if it is determined that the calorie intake is greater than the calorie requirement of the goal diet in step 110, and that the frequency of low-nutrient density foods (empty calories) is greater than two over a predetermined time then server 12 will search database 22, or third-party data source 20 for a food in the same category which has a medium nutrient density or higher, and is lower in calorie intake. For example, if donuts are part of dieter 14's natural diet as input in step 108 (assuming that the donut is the least-nutritional food as ranked), server 12 will search for foods in either the bread category, breakfast category, or snack category such as bagels, whole-grain cereals, or the like as a substitute for donuts. If for a food the calorie intake is greater than the calorie requirements, but the nutrient density is acceptable, then server 12 may determine that a smaller portion size is all that is required to gradually bring the natural diet in line with the goals of a diet for reaching the goals. Lastly, server 12 may determine that the substitution is to entirely remove the worst food without substitution.

Once the proposed replacement food is selected, server 12 first suggests a menu, which displays the natural diet menu to dieter 14 at their computer 16. It also provides a suggested menu with a proposed change as determined by the rules applied in rule 118 in step 116 with one or more suggested food substitutions to replace the least dieting goal oriented (worst) food with a more dieting goal oriented food.

In a preferred embodiment, the substitution may be displayed in several ways, it may be a text recommendation appended to the actual menu, it may be an e-mail to dieter 14, but in a preferred embodiment, the suggested revised menu is displayed in a side-by-side comparison to the current food menu of the user. Furthermore, the added component which is to be substituted for the existing menu, may be provided in a drop-down graphical user interface manner such that if dieter 14 does not wish to substitute a bagel for the donut as breakfast, dieter 14 will be offered the choice of the prescribed serving of whole-grain cereal, toast with appropriate spreads/toppings or the like so that dieter 14 can select their transition menu in accordance with the rule-determined nutrient and caloric parameters. Steps 108-118 are repeated until dieter 14's diet conforms to the goal; in our example until the dieter 14 actually consistently observes a low-fat, low-calorie diet sufficient to promote weight loss.

In the preferred embodiment, the lowest ranked food, i.e., the food which most drives dieter 14's diet away from the goal, is suggested to be substituted. However, it should be understood that it is also contemplated that a second or third lowest ranked food could also be suggested for substitution. It should be noted, that by gradually substituting for portions of the dieter's natural diet, an end result of the transition is a low-fat, low-calorie diet, by way of example, which incorporates a significant portion of the dieter's natural diet; assuming that the entire beginning diet is not substituted for. This is contrasted to a cold-turkey adoption of a pre-planned, pre-programmed diet menu as is now done with all diet programs. Furthermore, it should be noted, that a low-calorie, low-fat parameter was chosen by way of example, and the general model can be applied to any nutritional goal having components which can be tracked, such as weight maintenance with low cholesterol. It should also be noted and understood, that although only two parameters were discussed, the invention can be applied to multiple, and sometimes conflicting (high protein, low cholesterol) parameters to determine a diet as a function of nutritional information, and nutritional data, dieter characteristics and dieting goal. The goal is to transition the natural diet to the goal diet in a non-aggressive manner to prevent a shock to the dieter sufficient to cause the dieter to “fall off” the diet.

Thus while there have been shown, described and pointed out novel features of the present invention as applied to preferred embodiments thereof, it will be understood that various omissions and substitutions and change in the form and detail are contemplated so that the disclosed invention may be made by those skilled in the art without departing from the spirit and scope of the invention. It is the intention, therefore, to be limited only as indicated by the scope of the claims appended hereto. It is also to be understood that the following claims are intended to cover all of the generic and specific features of the invention herein described and all statements of the scope of the invention, which, as a matter of language, might be said to fall therebetween.

What is claimed is:

1. A method for transitioning a dieter's diet from a natural diet to a goal diet comprising the steps of:
storing characteristic data, said characteristic data including at least a diet goal;
tracking foods eaten for a time period sufficient to determine a natural diet;
determining a worst food eaten as a function of said diet goal and a replacement food for said worst food eaten;
said replacement food being more in keeping with said diet goal than said worst food as a function of food nutritional information and said dieter characteristic data;
developing an alternative menu in which said replacement food replaces said worst food; and
communicating said alternative menu to said dieter.

2. The method of claim 1, wherein said characteristic data includes at least one of an age, height, weight, activity level and medical condition of a dieter.

3. The method of claim 1, further comprising the step of providing a server, said server storing the characteristic data, ranking the elements of said food eaten, determining a worst food eaten and a replacement food, wherein said server determines whether said food eaten conforms to a diet which meets the diet goal and suggests a menu when said diet is in accordance with said diet goal.

4. The method of claim 1, further comprising the step of providing a server, said server storing the characteristic data, ranking the elements of said food eaten, determining a worst food eaten and a replacement food, wherein said server applies rules to said eaten food as a function of the nutritional value of said eaten food and said diet goal to determine said alternative menu.

5. The method claim 4 wherein said server weights each of said rules as a function of said diet goal.

6. The method of claim 1, wherein said replacement food corresponds to a smaller portion of a food of said natural diet.

7. The method of claim 1, wherein said replacement food corresponds to the elimination of a food from said natural diet.

8. The method of claim 1, wherein said foods are ranked from worst to best.

9. A system for transitioning a dieter's diet comprising:
a dieter computer; and a server in communication with said dieter computer, said server receiving dieter characteristic data input at said dieter computer and storing said dieter characteristic data, said dieter characteristic data including at least a diet, said server receiving dieter input foods corresponding to foods eaten for a period of time by said dieter and determining a dieter natural diet, said server determining a worst food eaten as a function of the diet goal and determining a replacement food for said worst food eaten, and communicating said replacement food to said dieter at said dieter computer.

10. The method of claim 9, wherein said server communicates with said dieter computer through the Internet.

11. The system of claim 9, wherein said replacement food is more in keeping with said diet goal than said worst food as a function of food nutritional information and said dieter characteristic data.

12. The method of claim 9, wherein said server develops an alternative menu in which said replacement food replaces said worst food.

13. The method of claim 9, further comprising a third-party data source in communication with said server, said nutritional information being stored at said third-party data source.

14. The system of claim 12, wherein said server applies logic rules to said eaten foods as a function of the nutritional value of said eaten food and said diet goal to determine said alternative menu.

15. The system of claim 12, wherein said server weights each of said rules as a function of said diet goal.
